SEQUENCE LISTING (8 sequences)

SEQ ID NO: 1 - DNA, 20 bp, Artificial Sequence
  Description of Artificial Sequence: Synthetic oligonucleotide
tccatgacgttcctgatgct

SEQ ID NO: 2 - DNA, 20 bp, Artificial Sequence
  Description of Artificial Sequence: Synthetic oligonucleotide
atcgactctcgagcgttctc

SEQ ID NO: 3-4 - skipped (2 entries)

SEQ ID NO: 5 - DNA, 31 bp, Artificial Sequence
  Description of Artificial Sequence: Primer
gtcagctagcgaggtgattcaggcagaagtt

SEQ ID NO: 6 - DNA, 25 bp, Artificial Sequence
  Description of Artificial Sequence: Primer
cagtgctagctcctatctcatagcc

SEQ ID NO: 7 - DNA, 66 bp, Homo sapiens
atggatgcaatgaagagagggctctgctgtgtgctgctgctgtgtggagcagtcttcgtttcggct

SEQ ID NO: 8 - protein, 22 aa, Homo sapiens
MDAMKRGLCCVLLLCGAVFVSA

(19) United States
(12) Patent Application Publication
(10) Pub. No.: US 2004/0082530 A1
(43) Pub. Date: Apr. 29, 2004

(54) Nucleic acid immunization

(76) Inventors: Connie S. Schmaljohn, Fort Detrick, MD (US); James T. Fuller, Middleton, WI (US)

Correspondence Address: Alisa Harbin, Chiron Corporation, P.O. Box 8097, Emeryville, CA, 94662, US

(21) Appl. No.: 10/411,205

(22) Filed: Apr. 11, 2003

Related U.S. Application Data
(60) Provisional application No. 60/371,416, filed on Apr. 11, 2002.

(51) Int. Cl. A61K 48/00

(52) U.S. Cl. 514/44

(57) ABSTRACT

Recombinant nucleic acid molecules are described. The molecules have a sequence or sequences encoding an antigen from Bacillus anthracis. Vectors and compositions containing these molecules are also described. Methods for eliciting an immune response using these molecules and compositions are also described.

### CROSS-REFERENCE TO RELATED APPLICATION

This application is related to U.S. provisional application serial No. 60/371,416, filed 11 Apr. 2002, from which priority is claimed pursuant to 35 U.S.C. §119(e)(1) and which application is incorporated herein by reference in its entirety.

### STATEMENT REGARDING FEDERALLY SPONSORED RESEARCH

This invention was made in connection with a CRADA awarded by the US Army Medical Research Institute of Infectious Diseases, which is an agency of the United States Government. The United States Government may have certain rights in the invention.

### TECHNICAL FIELD

The invention relates to the fields of molecular biology and immunology, and generally relates to nucleic acid immunization techniques. More specifically, the invention relates to polynucleotides encoding an antigen from Bacillus anthracis, and to nucleic acid immunization strategies employing such polynucleotides.

### BACKGROUND

Techniques for the injection of DNA and mRNA into mammalian tissue for the purposes of immunization against an expression product have been described in the art. The techniques, termed “nucleic acid immunization” herein, have been shown to elicit both humoral and cell-mediated immune responses. For example, sera from mice immunized with a DNA construct encoding the envelope glycoprotein, gp160, were shown to react with recombinant gp160 in immunoassays, and lymphocytes from the injected mice were shown to proliferate in response to recombinant gp120. Wang et al. (1993) Proc. Natl. Acad. Sci. USA 90:4156-4160. Similarly, mice immunized with a human growth hormone (hGH) gene demonstrated an antibody-based immune response. Tang et al. (1992) Nature 356: 152-154. Intramuscular injection of DNA encoding influenza nucleoprotein driven by a mammalian promoter has been shown to elicit a CD8+CTL response that can protect mice against subsequent lethal challenge with virus. Ulmer et al. (1993) Science 259:1745-1749. Immunohistochemical studies of the injection site revealed that the DNA was taken up by myeloblasts, and cytoplasmic production of viral protein could be demonstrated for at least 6 months.

### SUMMARY OF THE INVENTION

It is a primary object of the invention to provide a polynucleotide vaccine composition containing a nucleic acid sequence that encodes at least one antigen obtained or derived from Bacillus anthracis. Preferably, the nucleic acid sequence encodes the so-called Protective Antigen (“PA”) of Bacillus anthracis. The composition can be used as a reagent in various nucleic acid immunization strategies. In a related aspect of the invention, a composition is provided that contains a recombinant nucleic acid molecule that includes a nucleic acid sequence encoding an antigen obtained or derived from Bacillus anthracis linked to a second, heterologous nucleic acid sequence which encodes a peptide leader sequence. The second sequence is arranged in the recombinant molecule in a 5′ upstream position relative to the antigen sequence, and is linked to the antigen sequence to form a hybrid sequence. It is also a primary object of the invention to provide a method for eliciting an immune response against Bacillus anthracis in an immunized subject. The method entails transfecting cells of the subject with a polynucleotide vaccine composition according to the present invention, that is, a composition containing a sequence that encodes at least one Bacillus anthracis antigen. Expression cassettes and/or vectors containing any one of the nucleic acid molecules of the present invention can be used to transfect the cells, and transfection is carried out under conditions that permit expression of the antigens within the subject. The method may further entail one or more steps of administering at least one secondary composition to the subject.

The transfection procedure carried out during the immunization can be conducted either in vivo, or ex vivo (e.g., to obtain transfected cells which are subsequently introduced into the subject prior to carrying out the secondary immunization step). When in vivo transfection is used, the recombinant nucleic acid molecules can be administered to the subject by way of intramuscular or intradermal injection of plasmid DNA or other recombinant vector, preferably, administered to the subject using a particle-mediated delivery technique. Secondary vaccine compositions can include the same Bacillus anthracis antigen of interest, or other Bacillus anthracis antigens in the form of any suitable vaccine composition, for example, in the form of a recombinant Bacillus anthracis protein composition, or in the form of a nucleic acid vaccine composition.

Advantages of the present invention include, but are not limited to: (i) providing recombinant polynucleotides that encode a Bacillus anthracis antigen in mammalian cells; and (ii) use of these polynucleotides as reagents in nucleic acid immunization strategies to attain a broadly protective immune response against Bacillus anthracis infection and anthrax disease.

These and other objects, aspects, embodiments and advantages of the present invention will readily occur to those of ordinary skill in the art in view of the disclosure herein.

### BRIEF DESCRIPTION OF THE DRAWINGS

FIGS. 1A-1F depict the nucleic acid sequence for the Bacillus anthracis Protective Antigen (SEQ ID NO:3) and the predicted amino acid sequence for the expressed antigen (SEQ ID NO:4).

FIG. 2 depicts a functional map of the pWRG7077PA and pWRG7079 expression vector constructs used in the examples.

FIG. 3 depicts the anti-PA antibody titers in animals immunized with various anthrax vaccines per Example 3.

### DETAILED DESCRIPTION OF THE INVENTION

Before describing the present invention in detail, it is to be understood that this invention is not limited to particularly exemplified molecules, methods or process parameters as such may, of course, vary. It is also to be understood that the terminology used herein is for the purpose of describing particular embodiments of the invention only, and is not intended to be limiting. In addition, the practice of the present invention will employ, unless otherwise indicated, conventional methods of virology, microbiology, molecular biology, recombinant DNA techniques and immunology all of which are within the ordinary skill of the art. Such techniques are explained fully in the literature. See, e.g., Sambrook, et al., Molecular Cloning: A Laboratory Manual (2nd Edition, 1989); DNA Cloning: A Practical Approach, vol. I & II (D. Glover, ed.); Oligonucleotide Synthesis (N. Gait, ed., 1984); A Practical Guide to Molecular Cloning (1984); and Fundamental Virology, 2nd Edition, vol. I & II (B. N. Fields and D. M. Knipe, eds.).

All publications, patents and patent applications cited herein, whether supra or infra, are hereby incorporated by reference in their entirety.

It must be noted that, as used in this specification and the appended claims, the singular forms “a,” “an” and “the” include plural referents unless the content clearly dictates otherwise.

Definitions

Unless defined otherwise, all technical and scientific terms used herein have the same meaning as commonly understood by one of ordinary skill in the art to which the invention pertains. Although a number of methods and materials similar or equivalent to those described herein can be used in the practice of the present invention, the preferred materials and methods are described herein.

In describing the present invention, the following terms will be employed, and are intended to be defined as indicated below.

The term “nucleic acid immunization” is used herein to refer to the introduction of a nucleic acid molecule encoding one or more selected antigens into a host cell for the in vivo expression of the antigen or antigens. The nucleic acid molecule can be introduced directly into the recipient subject, such as by standard intramuscular or intradermal injection; transdermal particle delivery; inhalation; topically, or by oral, intranasal or mucosal modes of administration. The molecule alternatively can be introduced ex vivo into cells which have been removed from a subject. In this latter case, cells containing the nucleic acid molecule of interest are re-introduced into the subject such that an immune response can be mounted against the antigen encoded by the nucleic acid molecule. The nucleic acid molecules used in such immunization are generally referred to herein as “nucleic acid vaccines.”

By “core carrier” is meant a carrier on which a guest nucleic acid (e.g., DNA, RNA) is coated in order to impart a defined particle size as well as a sufficiently high density to achieve the momentum required for cell membrane penetration, such that the guest molecule can be delivered using particle-mediated techniques (see, e.g., U.S. Pat. No. 5,100,792). Core carriers typically include materials such as tungsten, gold, platinum, ferrite, polystyrene and latex. See e.g., Particle Bombardment Technology for Gene Transfer, (1994) Yang, N. ed., Oxford University Press, New York, N.Y. pages 10-11.

By “needleless syringe” is meant an instrument which delivers a particulate composition transdermally without the aid of a conventional needle to pierce the skin. Needleless syringes for use with the present invention are discussed throughout this document.

The term “transdermal” delivery intends intradermal (e.g., into the dermis or epidermis), transdermal (e.g., “percutaneous”) and transmucosal administration, i.e., delivery by passage of an agent into or through skin or mucosal tissue. See, e.g., Transdermal Drug Delivery: Developmental Issues and Research Initiatives, Hadgraft and Guy (eds.), Marcel Dekker, Inc., (1989); Controlled Drug Delivery: Fundamentals and Applications, Robinson and Lee (eds.), Marcel Dekker Inc., (1987); and Transdermal Delivery of Drugs, Vols. 1-3, Kydonieus and Bemer (eds.), CRC Press, (1987). Thus, the term encompasses delivery from a needleless syringe deliver as described in U.S. Pat. No. 5,630,796, as well as particle-mediated delivery as described in U.S. Pat. No. 5,865,796.

A “polypeptide” is used in it broadest sense to refer to a compound of two or more subunit amino acids, amino acid analogs, or other peptidomimetics. The subunits may be linked by peptide bonds or by other bonds, for example ester, ether, etc. As used herein, the term “amino acid” refers to either natural and/or unnatural or synthetic amino acids, including glycine and both the D or L optical isomers, and amino acid analogs and peptidomimetics. A peptide of three or more amino acids is commonly called an oligopeptide if the peptide chain is short. If the peptide chain is long, the peptide is typically called a polypeptide or a protein.

An “antigen” refers to any agent, generally a macromolecule, which can elicit an immunological response in an individual. The term may be used to refer to an individual macromolecule or to a homogeneous or heterogeneous population of antigenic macromolecules. As used herein, “antigen” is generally used to refer to a protein molecule or portion thereof which contains one or more epitopes. For purposes of the present invention, antigens can be obtained or derived from any appropriate source. Furthermore, for purposes of the present invention, an “antigen” includes a protein having modifications, such as deletions, additions and substitutions (generally conservative in nature) to the native sequence, so long as the protein maintains sufficient immunogenicity. These modifications may be deliberate, for example through site-directed mutagenesis, or may be accidental, such as through mutations of hosts which produce the antigens.

By “subunit vaccine” is meant a vaccine composition which includes one or more selected antigens but not all antigens, derived from or homologous to, an antigen from a pathogen of interest such as from a virus, bacterium, parasite or fungus. Such a composition is substantially free of intact pathogen cells or pathogenic particles, or is the lysate of such cells or particles. Thus, a “subunit vaccine” can be prepared from at least partially purified (preferably substantially purified) immunogenic polypeptides from the pathogen, or analogs thereof. Methods for obtaining an antigen to be included in a subunit vaccine can thus include standard purification techniques, recombinant production, or synthetic production.

An “immune response” against an antigen of interest is the development in an individual of a humoral and/or a cellular immune response to that antigen. For purposes of the present invention, a “humoral immune response” refers to an immune response mediated by antibody molecules, while a “cellular immune response” is one mediated by T-lymphocytes and/or other white blood cells.

The terms “nucleic acid molecule” and “polynucleotide” are used interchangeably herein and refer to a polymeric form of nucleotides of any length, either deoxyribonucleotides or ribonucleotides, or analogs thereof. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers.

A polynucleotide is typically composed of a specific sequence of four nucleotide bases: adenine (A); cytosine (C); guanine (G); and thymine (T) (uracil (U) for thymine (T) when the polynucleotide is RNA). Thus, the term nucleic acid sequence is the alphabetical representation of a polynucleotide molecule. This alphabetical representation can be input into databases in a computer having a central processing unit and used for bioinformatics applications such as functional genomics and homology searching.

A “vector” is capable of transferring nucleic acid sequences to target cells (e.g., viral vectors, non-viral vectors, particulate carriers, and liposomes). Typically, “vector construct,” “expression vector,” and “gene transfer vector,” mean any nucleic acid construct capable of directing the expression of a gene of interest and which can transfer gene sequences to target cells. Thus, the term includes cloning and expression vehicles, as well as viral vectors. A “plasmid” is a vector in the form of an extrachromosomal genetic element.

A nucleic acid sequence which “encodes” a selected antigen is a nucleic acid molecule which is transcribed (in the case of DNA) and translated (in the case of mRNA) into a polypeptide in vivo when placed under the control of appropriate regulatory sequences. The boundaries of the coding sequence are determined by a start codon at the 5′ (amino) terminus and a translation stop codon at the 3′ (carboxy) terminus. For the purposes of the invention, such nucleic acid sequences can include, but are not limited to, cDNA from viral, procaryotic or eucaryotic mRNA, genomic sequences from viral or procaryotic DNA or RNA, and even synthetic DNA sequences. A transcription termination sequence may be located 3′ to the coding sequence.

A “promoter” is a nucleotide sequence which initiates and regulates transcription of a polypeptide-encoding polynucleotide. Promoters can include inducible promoters (where expression of a polynucleotide sequence operably linked to the promoter is induced by an analyte, cofactor, regulatory protein, etc.), repressible promoters (where expression of a polynucleotide sequence operably linked to the promoter is repressed by an analyte, cofactor, regulatory protein, etc.), and constitutive promoters. It is intended that the term “promoter” or “control element” includes full-length promoter regions and functional (e.g., controls transcription or translation) segments of these regions.

“Operably linked” refers to an arrangement of elements wherein the components so described are configured so as to perform their usual function. Thus, a given promoter operably linked to a nucleic acid sequence is capable of effecting the expression of that sequence when the proper enzymes are present. The promoter need not be contiguous with the sequence, so long as it functions to direct the expression thereof. Thus, for example, intervening untranslated yet transcribed sequences can be present between the promoter sequence and the nucleic acid sequence and the promoter sequence can still be considered “operably linked” to the coding sequence.

“Recombinant” is used herein to describe a nucleic acid molecule (polynucleotide) of genomic, cDNA, semisynthetic, or synthetic origin which, by virtue of its origin or manipulation is not associated with all or a portion of the polynucleotide with which it is associated in nature and/or is linked to a polynucleotide other than that to which it is linked in nature. Two nucleic acid sequences which are contained within a single recombinant nucleic acid molecule are “heterologous” relative to each other when they are not normally associated with each other in nature.

Techniques for determining nucleic acid and amino acid “sequence identity” or “sequence homology” also are known in the art. Typically, such techniques include determining the nucleotide sequence of the mRNA for a gene and/or determining the amino acid sequence encoded thereby, and comparing these sequences to a second nucleotide or amino acid sequence. In general, “identity” refers to an exact nucleotide-to-nucleotide or amino acid-to-amino acid correspondence of two polynucleotides or polypeptide sequences, respectively. Two or more sequences (polynucleotide or amino acid) can be compared by determining their “percent identity.” The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman (1981) Advances in Applied Mathematics 2:482-489. This algorithm can be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov (1986) Nucl. Acids Res. 14(6):6745-6763. An exemplary implementation of this algorithm to determine percent identity of a sequence is provided by the Genetics Computer Group (Madison, Wis.) in the “BestFit” utility application. The default parameters for this method are described in the Wisconsin Sequence Analysis Package Program Manual, Version 8 (1995) (available from Genetics Computer Group, Madison, Wis.). A preferred method of establishing percent identity in the context of the present invention is to use the MPSRCH package of programs copyrighted by the University of Edinburgh, developed by John F. Collins and Shane S. Sturrok, and distributed by IntelliGenetics, Inc. (Mountain View, Calif.). From this suite of packages the Smith-Waterman algorithm can be employed where default parameters are used for the scoring table (for example, gap open penalty of 12, gap extension penalty of one, and a gap of six). From the data generated the “Match” value reflects “sequence identity.” Other suitable programs for calculating the percent identity or similarity between sequences are generally known in the art, for example, another alignment program is BLAST, used with default parameters. For example, BLASTN and BLASTP can be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs can be found at the following internet address: http://www.ncbi.nlm.gov/cgi-bin/BLAST.

Alternatively, homology can be determined by hybridization of polynucleotides under conditions which form stable duplexes between homologous regions, followed by digestion with single-stranded-specific nuclease(s), and size determination of the digested fragments. Two DNA, or two polypeptide sequences are “substantially homologous” to each other when the sequences exhibit at least about 80%-85%, preferably at least about 90%, and most preferably at least about 95%-98% sequence identity over a defined length of the molecules, as determined using the methods above. As used herein, substantially homologous also refers to sequences showing complete identity to the specified DNA or polypeptide sequence. DNA sequences that are substantially homologous can be identified in a Southern hybridization experiment under, for example, stringent conditions, as defined for that particular system. For example, stringent hybridization conditions can include 50% formamide, 5×Denhardt's Solution, 5×SSC, 0.1% SDS and 100 μg/ml denatured salmon sperm DNA and the washing conditions can include 2×SSC, 0.1% SDS at 37° C. followed by 1×SSC, 0.1% SDS at 68° C. Defining appropriate hybridization conditions is within the skill of the art. See, e.g., Sambrook et al., supra; DNA Cloning, supra; Nucleic Acid Hybridization, supra.

The term “adjuvant” intends any material or composition capable of specifically or non-specifically altering, enhancing, directing, redirecting, potentiating or initiating an antigen-specific immune response. Thus, coadministration of an adjuvant with an antigen may result in a lower dose or fewer doses of antigen being necessary to achieve a desired immune response in the subject to which the antigen is administered, or coadministration may result in a qualitatively and/or quantitatively different immune response in the subject. The effectiveness of an adjuvant can be determined by administering the adjuvant with a vaccine composition in parallel with vaccine composition alone to animals and comparing antibody and/or cellular-mediated immunity in the two groups using standard assays such as radioimmunoassay, ELISAs, CTL assays, and the like, all well known in the art. Typically, in a vaccine composition, the adjuvant is a separate moiety from the antigen, although a single molecule can have both adjuvant and antigen properties (e.g., cholera toxin).

An “adjuvant composition” intends any pharmaceutical composition containing an adjuvant. Adjuvant compositions can be delivered in the methods of the invention while in any suitable pharmaceutical form, for example, as a liquid, powder, cream, lotion, emulsion, gel or the like. However, preferred adjuvant compositions will be in particulate form. It is intended, although not always explicitly stated, that molecules having similar biological activity as wild-type or purified peptide or chemical adjuvants, and nucleic acid encoding adjuvant molecules can be used within the spirit and scope of the invention.

The terms “individual” and “subject” are used interchangeably herein to refer to any member of the subphylum cordata, including, without limitation, humans and other primates, including non-human primates such as chimpanzees and other apes and monkey species; farm animals such as cattle, sheep, pigs, goats and horses; domestic mammals such as dogs and cats; laboratory animals including rodents such as mice, rats and guinea pigs; birds, including domestic, wild and game birds such as chickens, turkeys and other gallinaceous birds, ducks, geese, and the like. The terms do not denote a particular age. Thus, both adult and newborn individuals are intended to be covered. The methods described herein are intended for use in any of the above vertebrate species, since the immune systems of all of these vertebrates operate similarly.

General Overview

The present invention provides novel nucleic acid molecules containing a sequence that encodes an antigen obtained or derived from Bacillis anthracis (B. anthracis). These molecules are useful in eliciting an immune response in a subject against B. anthracis. In particular, the present inventors have determined that, surprisingly, a nucleic acid immunization technique (e.g, particle-mediated delivery of core carrier particles coated with the nucleic acid molecules of the present invention) can be used to elicit an immune response against B. anthracis in an immunized subject, and that the resultant immune response provides protection against disease (anthrax) associated with infection by the B. anthracis pathogen.

The ability to elicit an immune response against B. anthracis in an immunized subject is useful in a wide variety of contexts, for example, generation of anti-B. anthracis antibodies (polyclonal and/or monocolonal) for use in passive immunization, diagnostics and research. In this regard, diagnostic and research reagents comprising antibodies against pathogens can be used for identification or confirmation of the presence of pathogens in test samples including biological samples, as well as for control reagents in immunological binding assays. In addition, the ability to elicit an immune response against B. anthracis in an immunized subject is useful for vaccination of individuals or populations who are at risk of infection by the B. anthracis pathogen.

Bacillus anthracis is responsible in man and animals for anthrax disease which can exist in intestinal, pulmonary or cutaneous form. The severe forms of anthrax disease may lead to death in infected individuals, and in the case of the pulmonary (inhaled) form of anthrax, the disease is often times 100% fatal. The pathogenicity of B. anthracis is expressed in two ways: a toxic effect made evident by the appearance of an edema; and a so-called lethal toxic effect which may lead to death in infected individuals. There are two main virulence factors possessed by B. anthracis, a poly-D-glutamic capsule that inhibits phagocytosis and two binary toxins which are formed from combinations selected from three protein factors. These two binary toxins possess a common cell receptor-binding component which, when combined with either one of the other two factors forms an active toxin. The binding component present in both of the active toxins is non-toxic and is involved in the binding of the B. anthracis toxins to cell membranes in an infected host. The other two protein factors constitute the active elements responsible for the manifestation of either the toxic effect of the edema type or the toxic effect with lethal character. These two active factors are termed edematogenic factor (“EF”) and lethal factor (“LF”). The non-toxic factor responsible for binding to cell membranes is called protective antigen (“PA”) since, during immunization assays, the capacity to confer active protection against the disease was initially attributed to this factor.

The three factors PA, LF and EF have been isolated and purified as reported by Fish et al. (1968) J. Bacteriol. 95:907-917, and the two toxins obtained by the combination of PA and LF and of PA and EF, have been characterized and described by Leppla et al. (1982) Proc. Natl. Acad. Sci. USA 79:3162-3166. The B. anthracis genes pag, cya and lef that encode the factors PA, EF and LF, respectively, are distributed on a plasmid termed “pX01” of B. anthracis, as described by Mikesell et al (1983) Infect. Immun. 39:371-376. In addition, the pag, cya and lef genes have been cloned and fully sequenced as described by Welkos et al. (1988) Gene 69:287-300; Escuyer et al. (1988) Gene 71:293-298; and Bragg et al. (1989) Gene 81:45-54.

The PA antigen, the non-toxic, cell-binding component of the above-described binary toxins, is the essential component of the currently available, licensed human vaccine called Anthrax Vaccine Adsorbed “AVA”, currently produced by Bioport, Inc. (Lansing, Mich.). The current vaccine is produced from sterile filtrates obtained from batch cultures of B. anthracis V770-NP1-R, a production strain derived from the Sterne strain (Sterne (1939) Onderstepoort J. Vet. Sci. Anim. Indust. 13:313-317) which, although avirulent, still needs to be handled as a Class III pathogen. The PA-containing filtrate is adsorbed onto aluminium hydroxide (see, e.g., Puziss et al. (1963) Appl. Microbiol. 11:330-334). This particular vaccine has been used for over 30 years to protect subjects at-risk of exposure to B. anthracis and was used recently to vaccinate US armed forces against anthrax.

In addition to the PA antigen, the AVA vaccine contains small amounts of the anthrax active toxin factors LF and EF, and a range of culture-derived proteins. These additional B. anthracis factors and contaminating culture proteins contribute to the recorded reactogenicity of the current vaccine in some individuals. For example, the AVA vaccine product results in a variety of adverse effects including: mild, moderate and severe local reactions at the site of injection; muscle aches; joint aches; rash; chills; fever; nausea; loss of appetite and malaise. The current vaccine is also expensive and requires a six-month vaccination course of between four and six inoculations. The efficacy of AVA is reportedly quite variable in different animal models. For example, AVA is poorly protective against inhalational anthrax in guinea pigs (Ivins et al. (1994) Vaccine 12:872-874), yet highly effective in rhesus monkeys (Pitt et al. (1996) Salisbury Med. Bull. Suppl 87: 130). Rabbit models are similar to rhesus monkeys, where AVA is highly efficacious against inhalational anthrax (Pitt et al. (1996) 96^{th} Ann. Meet. Am. Soc. Microbiol. E-70:278). It is now generally accepted that the guinea pig animal model is a poor model for human disease since the licensed vaccine (AVA) is only partially protective against parenteral anthrax challenge and poorly protective against a spore challenge (Pitt et al. (2001) Vaccine 19:4768-4773; Ivins et al. (1994) Vaccine 12:872-874; and Ivins et al. (1995) Vaccine 13:1779-1784). Furthermore, present evidence suggests that the current vaccine may not be effective against inhalation challenge with certain strains (Broster et al. (1990) Proceedings of the International Workshop on Anthrax, Apr. 11-13, 1989, Winchester UK., Salisbury Med. Bull. Supp. No. 68, pp. 91-92).

There has recently been a heightened concern regarding the possible use of B. anthracis as a bioterrorist or biowarfare agent, particularly in light of the revelation that Iraq produced and actually fielded B. anthracis spores for use in the Gulf War (Zilinskas (1997) J. Am. Med. Assoc. 278:418-424), and the bioterrorist anthrax attacks that led to the deaths of several US citizens after the Sep. 11, 2001 attack on New York. Accordingly, there remains an acute need for an effective anti-B. anthracis vaccine, particularly if it can provide adequate protection against the inhalational form of anthrax.

In addition to the V770-NP 1-R and Sterne production strains, a number of alternative procaryotic (bacterial) expression systems have been proposed for producing the current vaccine composition, including an Escherichia coli expression system (Vodkin et al. (1983) Cell 34:693-697), a Salmonella typhimurium expression system (Coulson et al. (1994) Vaccine 12:1395-1401), Bacillus subtilis expression systems (see, e.g., U.S. Pat. No. 6,267,966 to Baillie; Ivins et al. (1986) Infection and Immunity 54:537-542; and Baillie et al. (1994) Let. Appl. Microbiol. 19:225-227), and a number of recombinant Bacillus anthracis expression systems that are either asporogenic or unable to produce the LF or EF toxins (see, e.g., U.S. Pat. No. 5,840,312 to Mock et al. and U.S. Pat. No. 6,316,006 to Worsham et al.). However, these alternative bacterial expression systems may fail to provide commercially viable production levels, or may introduce additional components into the final composition, thereby altering or affecting the final vaccine product.

As noted above, the present invention relates to the surprising discovery that a nucleic acid immunization technique can be used to provide a robust, B. anthracis-specific immune response, and that this immune response is able to provide significant vaccine protection against anthrax disease in a rabbit model that is an excellent predictor of human vaccine efficacy. Thus, in one embodiment of the invention, a polynucleotide vaccine composition is provided, wherein the composition contains a nucleic acid sequence encoding an antigen obtained or derived from one of the major B. anthracis protein factors. Preferably, the nucleic acid sequence encodes an antigen obtained or derived from the B. anthracis PA antigen sequence, and even more preferably, the nucleic acid sequence encodes a substantially full-length PA antigen, or a protein or peptide that is substantially homologous to the full-length PA antigen.

The polynucleotide vaccine compositions of the invention can be used as standalone vaccines, or as part of a multi-component vaccine composition. For example, in a multi-component vaccine composition, the present nucleic acid molecules are combined with additional nucleic acid molecules encoding additional B. anthracis antigens, for example, molecules containing sequences that encode portions of the EF or LF toxin antigens. Alternatively, the multi-component vaccine composition may contain the conventional (AVA) anthrax vaccine antigen. These additional components may complement the efficacy of the present polynucleotide vaccine to provide protective immune responses in vaccinated subjects. Thus, the invention provides more effective vaccines and methods of immunization against infection with B. anthracis.

Polynucleotides

In one embodiment, a recombinant polynucleotide vaccine composition is provided. The composition includes one or more nucleic acid molecules that contain a sequence encoding an antigen obtained or derived from B. anthracis. In one particular embodiment, a nucleic acid molecule is provided which contains a polynucleotide sequence encoding the PA antigen.

The complete gene sequence for the B. anthracis PA antigen is known (Welkos et al. (1988) Gene 69:287-300) and publically available. Active variants and functional homologues of this antigen sequence may also be used in the compositions and methods of the present invention. Sequences encoding the selected antigen are typically inserted into an appropriate vector (e.g., a plasmid backbone) using known techniques and as described below in the Examples.

More particularly, the sequence or sequences encoding the selected B. anthracis antigen of interest can be obtained and/or prepared using known methods. For example, substantially pure antigen preparations can be obtained using standard molecular biological tools. That is, the published PA gene sequence can be used to design suitable primers that can be used to obtain the complete PA gene sequence from a suitable B. anthracis strain, for example, from the Steme strain, or from a recombinant vector known to include the PA antigen sequence. See, e.g., Sambrook et al., supra, for a description of techniques used to obtain and isolate nucleic acid molecules. Polynucleotide sequences can also be produced synthetically, rather than cloned.

The most convenient method for isolating specific nucleic acid molecules is by the polymerase chain reaction (PCR). Mullis et al. (1987) Methods Enzymol. 155:335-350. This technique uses DNA polymerase, usually a thermostable DNA polymerase, to replicate a desired region of DNA. The region of DNA to be replicated is identified by oligonucleotides of specified sequence complementary to opposite ends and opposite strands of the desired DNA to prime the replication reaction. The product of the first round of replication is itself a template for subsequent replication, thus repeated successive cycles of replication result in geometric amplification of the DNA fragment delimited by the primer pair used.

These same techniques can be used to obtain sequences encoding other B. anthracis antigens. The relative ease of producing and purifying nucleic acid constructs facilitates the generation of combination vaccines, for example, polynucleotide vaccine compositions that contain one or more nucleic acid molecules containing a sequence encoding the PA antigen in combination with other B. anthracis sequences.

In some molecules, an ancillary sequence can be included which provides for secretion of an attached hybrid antigen molecule from a mammalian cell. Such secretion leader sequences are known to those skilled in the art, and include, for example, the tissue plasminogen activator (TPA) leader signal sequence.

Once the relevant sequences for the B. anthracis antigen of interest and, alternatively, sequences encoding other B. anthracis antigens such as fragments of EF or LF antigens and/or ancillary sequences such as a leader sequence, have been obtained, they can be linked together to provide one or more contiguous nucleic acid molecules using standard cloning or molecular biology techniques. More particularly, after sequence information for the antigen of interest has been obtained, it can be combined with other sequences to form a hybrid sequence, or handled separately. In hybrid sequences, the various antigen and ancillary sequences can be positioned in any manner relative to each other, and be included in a single molecule in any number ways, for example, as a single copy, randomly repeated in the molecule as multiple copies, or included in the molecule as multiple tandem repeats or otherwise ordered repeat motifs.

Although any number of routine molecular biology techniques can be used to construct such recombinant nucleic acid molecules, one convenient method entails using one or more unique restriction sites in a shuttle or cloning vector (or inserting one or more unique restriction sites into a suitable vector sequence) and standard cloning techniques to direct the B. anthracis antigen sequence or sequences to particular target locations within a vector.

Alternatively, hybrid molecules can be produced synthetically rather than cloned. The nucleotide sequence can be designed with the appropriate codons for the particular amino acid sequence desired. In general, one will select preferred codons for the intended host in which the sequence will be expressed. The complete sequence can then be assembled from overlapping oligonucleotides prepared by standard methods and assembled into a complete coding sequence. See, e.g., Edge (1981) Nature 292:756; Nambair et al. (1984) Science (1984) 223:1299; Jay et al. (1984) J. Biol. Chem. 259:6311.

Once the relevant B. anthracis antigen sequence (e.g., PA and, optionally, additional sequences that encode other B. anthracis antigens and/or ancillary sequences such as leader sequences) has been obtained or constructed, it can be inserted into a vector which includes control sequences operably linked to the inserted sequence or sequences, thus providing expression cassettes that allow for expression of the antigen in vivo in a targeted subject species, most suitably a mammalian subject.

Typical promoters for mammalian cell expression include the SV40 early promoter, a CMV promoter such as the CMV immediate early promoter, the mouse mammary tumor virus LTR promoter, the adenovirus major late promoter (Ad MLP), and other suitably efficient promoter systems. Nonviral promoters, such as a promoter derived from the murine metallothionein gene, may also be used for mammalian expression. Inducible, repressible or otherwise controllable promoters may also be used. Typically, transcription termination and polyadenylation sequences will also be present, located 3′ to each translation stop codon. Preferably, a sequence for optimization of initiation of translation, located 5′ to each coding sequence, is also present. Examples of transcription terminator/polyadenylation signals include those derived from SV40, as described in Sambrook et al., supra, as well as a bovine growth hormone terminator sequence. Introns, containing splice donor and acceptor sites, may also be designed into the expression cassette.

In addition, enhancer elements may be included within the expression cassettes in order to increase expression levels. Examples of suitable enhancers include the SV40 early gene enhancer (Dijkema et al. (1985) EMBO J. 4:761), the enhancer/promoter derived from the long terminal repeat (LTR) of the Rous Sarcoma Virus (Gorman et al. (1982) Proc. Natl. Acad. Sci. USA 79:6777), and elements derived from human or murine CMV (Boshart et al. (1985) Cell 41:521), for example, elements included in the CMV intron A sequence.

Adjuvants

Although not required, the polynucleotide vaccine compositions of the present invention may effectively be used with any suitable adjuvant or combination of adjuvants. For example, suitable adjuvants include, without limitation, adjuvants formed from aluminum salts (alum), such as aluminum hydroxide, aluminum phosphate, aluminum sulfate, etc; oil-in-water and water-in-oil emulsion formulations, such as Complete Freunds Adjuvants (CFA) and Incomplete Freunds Adjuvant (IFA); adjuvants formed from bacterial cell wall components such as adjuvants including lipopolysaccharides (e.g., lipid A or monophosphoryl lipid A (MPL), Imoto et al. (1985) Tet. Lett. 26:1545-1548), trehalose dimycolate (TDM), and cell wall skeleton (CWS); heat shock protein or derivatives thereof; adjuvants derived from ADP-ribosylating bacterial toxins, including diphtheria toxin (DT), pertussis toxin (PT), cholera toxin (CT), the E. coli heat-labile toxins (LT1 and LT2), Pseudomonas endotoxin A, Pseudomonas exotoxin S, B. cereus exoenzyme, B. sphaericus toxin, C. botulinum C2 and C3 toxins, C. limosum exoenzyme, as well as toxins from C. perfringens, C. spiriforma and C. difficile, Staphylococcus aureus EDIN, and ADP-ribosylating bacterial toxin mutants such as CRM₁₉₇, a non-toxic diphtheria toxin mutant (see, e.g., Bixler et al. (1989) Adv. Exp. Med. Biol. 251:175; and Constantino et al. (1992) Vaccine); saponin adjuvants such as Quil A (U.S. Pat. No. 5,057,540), or particles generated from saponins such as ISCOMs (immunostimulating complexes); chemokines and cytokines, such as interleukins (e.g., IL-1, IL-2, IL4, IL-5, IL-6, IL-7, IL-8, IL-12, etc.), interferons (e.g., gama interferon), macrophage colony stimulating factor (M-CSF), tumor necrosis factor (TNF), defensins 1 or 2, RANTES, MIP1-α and MIP-2, etc; muramyl peptides such as N-acetyl-muramyl-L-threonyl-D-isoglutamine (thr-MDP), N-acetyl-normuramyl-^{L}-alanyl-^{D}-isoglutamine (nor-MDP), N-acetylmuramyl-^{L}-alanyl-^{D}-isoglutaminyl-^{L}-alanine-2-(1′-2′-dipalmitoyl-sn-glycero-3 huydroxyphosphoryloxy)-ethylamine (MTP-PE) etc.; adjuvants derived from the CpG family of molecules, CpG dinucleotides and synthetic oligonucleotides which comprise CpG motifs (see, e.g., Krieg et al. Nature (1995) 374:546, Medzhitov et al. (1997) Curr. Opin. Immunol. 9:4-9, and Davis et al. J. Immunol. (1998) 160:870-876) such as TCCATGACGTTCCTGATGCT (SEQ ID NO:1) and ATCGACTCTCGAGCGTTCTC (SEQ ID NO:2); and synthetic adjuvants such as PCPP (Poly[di(carboxylatophenoxy)phosphazene) (Payne et al. Vaccines (1998) 16:92-98). Such adjuvants are commercially available from a number of distributors such as Accurate Chemicals; Ribi Immunechemicals, Hamilton, Mont.; GIBCO; Sigma, St. Louis, Mo. Preferred adjuvants are those derived from ADP-ribosylating bacterial toxins, with cholera toxin and heat labile toxins being most preferred. Oligonucleotides containing a CpG motif are also preferred. Other preferred adjuvants are those provided in nucleic acid form, for example nucleic acid sequences that encode chemokines and cytokines, such as interleukins (e.g., IL-1, IL-2, IL-4, IL-5, IL-6, IL-7, IL-8, IL-12, etc.), interferons (e.g., gama interferon), macrophage colony stimulating factor (M-CSF), tumor necrosis factor (TNF), defensins 1 or 2, RANTES, MIP1-α and MIP-2 molecules.

The adjuvant may delivered individually or delivered in a combination of two or more adjuvants. In this regard, combined adjuvants may have an additive or a synergistic effect in promoting a desired immune response. A synergistic effect is one where the result achieved by combining two or more adjuvants is greater than one would expect than by merely adding the result achieved with each adjuvant when administered individually. A preferred adjuvant combination is an adjuvant derived from an ADP-ribosylating bacterial toxin and a synthetic oligonucleotide comprising a CpG motif. A particularly preferred combination comprises cholera toxin and the oligonucleotide ATCGACTCTCGAGCGTTCTC (SEQ ID NO:2).

Unfortunately, a majority of the above-referenced adjuvants are known to be highly toxic, and are thus generally considered too toxic for human use. It is for this reason that the only ubiquitous adjuvant currently approved for human usage is alum, an aluminum salt composition. Nevertheless, a number of the above adjuvants are commonly used in animals and thus suitable for numerous intended subjects, and several are undergoing preclinical and clinical studies for human use. However, as discussed herein above, the adjuvants employed in the present invention are preferably rendered into particulate form for transdermal delivery using a powder injection method. Surprisingly, it has been found that adjuvants which are generally considered too toxic for human use may be rendered into particulate form and administered with a powder injection technique without concomitant toxicity problems. Without being bound by a particular theory, it appears that delivery of adjuvants to the skin, using transdermal delivery methods (powder injection), allows interaction with Langerhans cells in the epidermal layer and dendritic cells in the cutaneous layer of the skin. These cells are important in initiation and maintenance of an immune response. Thus, an enhanced adjuvant effect can be obtained by targeting delivery to or near such cells. Moreover, transdermal delivery of adjuvants in the practice of the invention may avoid toxicity problems because (1) the top layers of the skin are poorly vascularized, thus the amount of adjuvant entering the systemic circulation is reduced which reduces the toxic effect; (2) skin cells are constantly being sloughed, therefore residual adjuvant is eliminated rather than absorbed; and (3) substantially less adjuvant can be administered to produce a suitable adjuvant effect (as compared with adjuvant that is delivered using conventional techniques such as intramuscular injection).

Once selected, one or more adjuvant can be provided in a suitable pharmaceutical form for parenteral delivery, the preparation of which forms are well within the general skill of the art. See, e.g., Remington's Pharmaceutical Sciences (1990) Mack Publishing Company, Easton, Pa., 18th edition. Alternatively, the adjuvant can be rendered into particulate form as described in detail below. The adjuvant(s) will be present in the pharmaceutical form in an amount sufficient to bring about the desired effect, that is, either to enhance the response against the coadministered antigen of interest, and/or to direct an immune response against the antigen of interest. Generally about 0.1 μg to 1000 μg of adjuvant, more preferably about 1 μg to 500 μg of adjuvant, and more preferably about 5 μg to 300 μg of adjuvant will be effective to enhance an immune response of a given antigen. Thus, for example, for CpG, doses in the range of about 0.5 to 50 μg, preferably about 1 to 25 μg, and more preferably about 5 to 20 μg, will find use with the present methods. For cholera toxin, a dose in the range of about 0.1 μg to 50 μg, preferably about 1 μg to 25 μg, and more preferably about 5 μg to 15 μg will find use herein. Similarly, for alum or PCPP, a dose in the range of about 2.5 μg to 500 μg, preferably about 25 to 250 μg, and more preferably about 50 to 150 μg, will find use herein. For MPL, a dose in the range of about 1 to 250 μg, preferably about 20 to 150 μg, and more preferably about 40 to 75 μg, will find use with the present methods.

Doses for other adjuvants can readily be determined by one of skill in the art using routine methods. The amount to administer will depend on a number of factors including the nature of the B. anthracis antigen.

Administration of Polynucleotides

Once complete, the polynucleotide constructs are used for nucleic acid immunization using standard gene delivery protocols. Numerous methods for delivering nucleic acid molecules are known in the art. The nucleic acid molecules of the present invention (present in a suitable expression cassette) can thus be delivered either directly to a subject or, alternatively, delivered ex vivo to cells derived from the subject whereafter the cells are reimplanted in the subject. The most convenient way to delivery the polynucleotide constructs is in a plasmid (DNA) vector. Alternatively, a viral vector can be used. A number of viral based systems have been developed for transfecting mammalian cells. For example, a selected nucleic acid molecule containing a sequence or sequences encoding B. anthracis antigen(s) can be inserted into a vector and packaged as retroviral particles using techniques known in the art. The recombinant virus can then be isolated and delivered to cells of the subject either in vivo or ex vivo. A number of retroviral systems have been described (U.S. Pat. No. 5,219,740; Miller et al. (1989) BioTechniques 7:980-990; Miller, A. D. (1990) Human Gene Therapy 1:5-14; and Burns et al. (1993) Proc. Natl. Acad. Sci. USA 90:8033-8037.

A number of adenovirus vectors have also been described (Haj-Ahmad et al. (1986) J. Virol. 57:267-274; Bett et al. (1993) J. Virol. 67:5911-5921; Mittereder et al. (1994) Human Gene Therapy 5:717-729; and Rich et al. (1993) Human Gene Therapy 4:461-476). Additionally, various adeno-associated virus (AAV) vector systems have been developed. AAV vectors can be readily constructed using techniques well known in the art. See, e.g., U.S. Pat. Nos. 5,173,414 and 5,139,941; International Publication Nos. WO 92/01070 (published 23 Jan. 1992) and WO 93/03769 (published 4 Mar. 1993); Lebkowski et al. (1988) Molec. Cell. Biol. 8:3988-3996; Vincent et al. (1990) Vaccines 90 (Cold Spring Harbor Laboratory Press); Carter, B. J. (1992) Current Opinion in Biotechnology 3:533-539; Muzyczka, N. (1992) Current Topics in Microbiol. and Immunol. 158:97-129; and Kotin, R. M. (1994) Human Gene Therapy 5:793-801. Additional viral vectors which will find use for delivering the recombinant nucleic acid molecules of the present invention include, but are not limited to, those derived from the pox family of viruses, including vaccinia virus and avian poxvirus.

Conventional Pharmaceutical Preparations

Formulation of a preparation comprising the above-described recombinant polynucleotide vaccines, with or without addition of an adjuvant composition, can be carried out using standard pharmaceutical formulation chemistries and methodologies all of which are readily available to the ordinarily skilled artisan. For example, compositions containing one or more nucleic acid sequences (e.g., present in a suitable vector form such as a DNA plasmid) can be combined with one or more pharmaceutically acceptable excipients or vehicles to provide a liquid preparation.

Auxiliary substances, such as wetting or emulsifying agents, pH buffering substances and the like, may be present in the excipient or vehicle. These excipients, vehicles and auxiliary substances are generally pharmaceutical agents that do not induce an immune response in the individual receiving the composition, and which may be administered without undue toxicity. Pharmaceutically acceptable excipients include, but are not limited to, liquids such as water, saline, polyethyleneglycol, hyaluronic acid, glycerol and ethanol. Pharmaceutically acceptable salts can also be included therein, for example, mineral acid salts such as hydrochlorides, hydrobromides, phosphates, sulfates, and the like; and the salts of organic acids such as acetates, propionates, malonates, benzoates, and the like. It is also preferred, although not required, that the preparation will contain a pharmaceutically acceptable excipient that serves as a stabilizer, particularly for peptide, protein or other like molecules if they are to be included in a (combined) vaccine composition. Examples of suitable carriers that also act as stabilizers for peptides include, without limitation, pharmaceutical grades of dextrose, sucrose, lactose, trehalose, mannitol, sorbitol, inositol, dextran, and the like. Other suitable carriers include, again without limitation, starch, cellulose, sodium or calcium phosphates, citric acid, tartaric acid, glycine, high molecular weight polyethylene glycols (PEGs), and combination thereof. A thorough discussion of pharmaceutically acceptable excipients, vehicles and auxiliary substances is available in REMINGTON'S PHARMACEUTICAL SCIENCES (Mack Pub. Co., N.J. 1991), incorporated herein by reference.

Certain facilitators of nucleic acid uptake and/or expression (“transfection facilitating agents”) can also be included in the compositions, for example, facilitators such as bupivacaine, urea, cardiotoxin and sucrose, and transfection facilitating vehicles such as liposomal or lipid preparations that are routinely used to deliver nucleic acid molecules. Anionic and neutral liposomes are widely available and well known for delivering nucleic acid molecules (see, e.g., Liposomes: A Practical Approach, (1990) RPC New Ed., IRL Press). Cationic lipid preparations are also well known vehicles for use in delivery of nucleic acid molecules. Suitable lipid preparations include DOTMA (N-[1-(2,3-dioleyloxy)propyl]-N,N,N-trimethylammonium chloride), available under the tradename Lipofectin#, and DOTAP (1,2-bis(oleyloxy)-3(trimethylammonio)propane), see, e.g., Felgner et al. (1987) Proc. Natl. Acad. Sci. USA 84:7413-7416; Malone et al. (1989) Proc. Natl. Acad. Sci. USA 86:6077-6081; U.S. Pat. Nos. 5,283,185 and 5,527,928, and International Publication Nos WO 90/11092, WO 91/15501 and WO 95/26356. These cationic lipids may preferably be used in association with a neutral lipid, for example DOPE (dioleyl phosphatidylethanolamine). Still further transfection-facilitating compositions that can be added to the above lipid or liposome preparations include spermine derivatives (see, e.g., International Publication No. WO 93/18759) and membrane-permeabilizing compounds such as GALA, Gramicidine S and cationic bile salts (see, e.g., International Publicaiton No. WO 93/19768).

Alternatively, the nucleic acid molecules of the present invention may be encapsulated, adsorbed to, or associated with, particulate carriers. Suitable particulate carriers include those derived from polymethyl methacrylate polymers, as well as PLG microparticles derived from poly(lactides) and poly(lactide-co-glycolides). See, e.g., Jeffery et al. (1993) Pharm. Res. 10:362-368. Other particulate systems and polymers can also be used, for example, polymers such as polylysine, polyarginine, polyornithine, spermine, spermidine, as well as conjugates of these molecules.

The formulated vaccine compositions will include a polynucleotide containing a sequence that encodes the selected B. anthracis PA antigen or antigens of interest in an amount sufficient to mount an immunological response. An appropriate effective amount can be readily determined by one of skill in the art. Such an amount will fall in a relatively broad range that can be determined through routine trials. For example, immune responses have been obtained using as little as 1 μg of DNA, while in other administrations, up to 2 mg of DNA has been used. It is generally expected that an effective dose of the polynucleotide will fall within a range of about 10 μg to 1000 μg, however, doses above and below this range may also be found effective. The compositions may thus contain from about 0.1% to about 99.9% of the polynucleotide molecules and can be administered directly to the subject or, alternatively, delivered ex vivo, to cells derived from the subject, using methods known to those skilled in the art

Administration of Conventional Preparations

Once suitably formulated, these vaccine compositions can be administered to a subject in vivo using a variety of known routes and techniques. For example, the liquid preparations can be provided as an injectable solution, suspension or emulsion and administered via parenteral, subcutaneous, intradermal, intramuscular, intravenous injection using a conventional needle and syringe, or using a liquid jet injection system. Liquid preparations can also be administered topically to skin or mucosal tissue, or provided as a finely divided spray suitable for respiratory or pulmonary administration. Other modes of administration include oral administration, suppositories, and active or passive transdermal delivery techniques.

Alternatively, the vaccine compositions can be administered ex vivo, for example delivery and reimplantation of transformed cells into a subject are known (e.g., dextran-mediated transfection, calcium phosphate precipitation, electroporation, and direct microinjection of into nuclei).

Coated Particle Pharmaceutical Preparations

In a preferred embodiment, the polynucleotide vaccine compositions (e.g., a DNA vaccine), whether or not combined with conventional B. anthracis vaccine compositions (e.g., the AVA vaccine product) and/or adjuvants are delivered using carrier particles. Particle-mediated methods for delivering such nucleic acid preparations are known in the art. Thus, once prepared and suitably purified, the above-described nucleic acid molecules and/or adjuvants can be coated onto carrier particles (e.g., core carriers) using a variety of techniques known in the art. Carrier particles are selected from materials which have a suitable density in the range of particle sizes typically used for intracellular delivery from a particle-mediated delivery device. The optimum carrier particle size will, of course, depend on the diameter of the target cells. Alternatively, colloidal gold particles can be used wherein the coated colloidal gold is administered (e.g., injected) into tissue (e.g., skin or muscle) and subsequently taken-up by immune-competent cells.

For the purposes of the invention, tungsten, gold, platinum and iridium carrier particles can be used. Tungsten and gold particles are preferred. Tungsten particles are readily available in average sizes of 0.5 to 2.0 μm in diameter. Although such particles have optimal density for use in particle acceleration delivery methods, and allow highly efficient coating with DNA, tungsten may potentially be toxic to certain cell types. Gold particles or microcrystalline gold (e.g., gold powder A1570, available from Engelhard Corp., East Newark, N.J.) will also find use with the present methods. Gold particles provide uniformity in size (available from Alpha Chemicals in particle sizes of 1-3 82 m, or available from Degussa, South Plainfield, N.J. in a range of particle sizes including 0.95 μm) and reduced toxicity. Microcrystalline gold provides a diverse particle size distribution, typically in the range of 0.1-5 μm. However, the irregular surface area of microcrystalline gold provides for highly efficient coating with nucleic acids.

A number of methods are known and have been described for coating or precipitating DNA or RNA onto gold or tungsten particles. Most such methods generally combine a predetermined amount of gold or tungsten with plasmid DNA, CaCl₂ and spermidine. The resulting solution is vortexed continually during the coating procedure to ensure uniformity of the reaction mixture. After precipitation of the nucleic acid, the coated particles can be transferred to suitable membranes and allowed to dry prior to use, coated onto surfaces of a sample module or cassette, or loaded into a delivery cassette for use in particular particle-mediated delivery instruments.

Peptides (e.g., a B. anthracis recombinant PA protein subunit vaccine, and/or a protein or peptide adjuvant moiety), can also be coated onto suitable carrier particles, e.g., gold or tungsten. For example, peptides can be attached to the carrier particle by simply mixing the two components in an empirically determined ratio, by ammonium sulfate precipitation or solvent precipitation methods familiar to those skilled in the art, or by chemical coupling of the peptide to the carrier particle. The coupling of L-cysteine residues to gold has been previously described (Brown et al., Chemical Society Reviews 9:271-311 (1980)). Other methods include, for example, dissolving the peptide antigen in absolute ethanol, water, or an alcohol/water mixture, adding the solution to a quantity of carrier particles, and then drying the mixture under a stream of air or nitrogen gas while vortexing. Alternatively, the peptide antigens can be dried onto carrier particles by centrifugation under vacuum. Once dried, the coated particles can be resuspended in a suitable solvent (e.g., ethyl acetate or acetone), and triturated (e.g., by sonication) to provide a substantially uniform suspension.

Administration of Coated Particles

Following their formation, carrier particles coated with the nucleic acid preparations and, alternatively, adjuvants and/or B. anthracis peptide or protein antigen preparations, can be delivered to a subject using particle-mediated delivery techniques.

Various particle acceleration devices suitable for particle-mediated delivery are known in the art, and are all suited for use in the practice of the invention. Current device designs employ an explosive, electric or gaseous discharge to propel coated carrier particles toward target cells. The coated carrier particles can themselves be releasably attached to a movable carrier sheet, or removably attached to a surface along which a gas stream passes, lifting the particles from the surface and accelerating them toward the target. An example of a gaseous discharge device is described in U.S. Pat. No. 5,204,253. An explosive-type device is described in U.S. Pat. No. 4,945,050. One example of an electric discharge-type particle acceleration apparatus is described in U.S. Pat. No. 5,120,657. Another electric discharge apparatus suitable for use herein is described in U.S. Pat. No. 5,149,655. The disclosure of all of these patents is incorporated herein by reference in their entireties.

If desired, these particle acceleration devices can be provided in a pre-loaded condition containing a suitable dosage of the coated carrier particles comprising the polynucleotide vaccine composition, with or without additional influenza vaccine compositions and/or a selected adjuvant component. The loaded syringe can be packaged in a hermetically sealed container.

The coated particles are administered to the subject to be treated in a manner compatible with the dosage formulation, and in an amount that will be effective to bring about a desired immune response. The amount of the composition to be delivered which, in the case of nucleic acid molecules is generally in the range of from 0.001 to 1000 μg, more preferably 0.01 to 10.0 μg of nucleic acid molecule per dose, and in the case of peptide or protein molecules is 1 μg to 5 μg, more preferably 1 to 50 μg of peptide, depends on the subject to be treated. The exact amount necessary will vary depending on the age and general condition of the individual being immunized and the particular nucleotide sequence or peptide selected, as well as other factors. An appropriate effective amount can be readily determined by one of skill in the art upon reading the instant specification.

Particulate Pharmaceutical Preparations

Alternatively, the polynucleotides of the present invention (as well as one or more selected adjuvant and/or conventional B. anthracis recombinant PA protein subunit vaccine compositions) can also be formulated as a particulate composition. More particularly, formulation of particles comprising the antigen and/or adjuvant of interest can be carried out using standard pharmaceutical formulation chemistries. For example, the polynucleotides and/or adjuvants can be combined with one or more pharmaceutically acceptable excipient or vehicle to provide a suitable vaccine composition.

The formulated compositions are then prepared as particles using standard techniques, such as by simple evaporation (air drying), vacuum drying, spray drying, freeze drying (lyophilization), spray-freeze drying, spray coating, precipitation, supercritical fluid particle formation, and the like. If desired, the resultant particles can be densified using the techniques described in International Publication No. WO 97/48485, incorporated herein by reference.

These methods can be used to obtain nucleic acid particles having a size ranging from about 0.01 to about 250 μm, preferably about 10 to about 150 μm, and most preferably about 20 to about 60 μm; and a particle density ranging from about 0.1 to about 25 g/cm³, and a bulk density of about 0.5 to about 3.0 g/cm³, or greater.

Similarly, particles of selected adjuvants having a size ranging from about 0.1 to about 250 μm, preferably about 0.1 to about 150 μm, and most preferably about 20 to about 60 μm; a particle density ranging from about 0.1 to about 25 g/cm³, and a bulk density of preferably about 0.5 to about 3.0 g/cm³, and most preferably about 0.8 to about 1.5 g/cm³ can be obtained.

Single unit dosages or multidose containers, in which the particles may be packaged prior to use, can comprise a hermetically sealed container enclosing a suitable amount of the particles comprising the antigen of interest and/or the selected adjuvant (e.g., the vaccine composition). The particulate compositions can be packaged as a sterile formulation, and the hermetically sealed container can thus be designed to preserve sterility of the formulation until use in the methods of the invention. If desired, the containers can be adapted for direct use in a needleless syringe system. Such containers can take the form of capsules, foil pouches, sachets, cassettes, and the like. Appropriate needleless syringes are described herein.

The container in which the particles are packaged can further be labelled to identify the composition and provide relevant dosage information. In addition, the container can be labelled with a notice in the form prescribed by a governmental agency, for example the Food and Drug Administration, wherein the notice indicates approval by the agency under Federal law of the manufacture, use or sale of the antigen, adjuvant (or vaccine composition) contained therein for human administration.

Administration of Particulate Compositions

Following their formation, the particulate composition (e.g., powder) can be delivered transdermally to the subject's tissue using a suitable transdermal delivery technique. Various particle acceleration devices suitable for transdermal delivery of the substance of interest are known in the art, and will find use in the practice of the invention. A particularly preferred transdermal delivery system employs a needleless syringe to fire solid drug-containing particles in controlled doses into and through intact skin and tissue. See, e.g., U.S. Pat. No. 5,630,796 to Bellhouse et al. which describes a needleless syringe (also known as “the PowderJect® needleless syringe device”). Other needleless syringe configurations are known in the art and are described herein.

The particulate compositions (comprising the antigen of interest and/or a selected adjuvant) can be administered using a transdermal delivery technique. Preferably, the particulate compositions will be delivered via a powder injection method, e.g., delivered from a needleless syringe system such as those described in International Publication Nos. WO 94/24263, WO 96/04947, WO 96/12513, and WO 96/20022, all of which are incorporated herein by reference. Delivery of particles from such needleless syringe systems is typically practised with particles having an approximate size generally ranging from 0.1 to 250 μm, preferably ranging from about 10-70 μm. Particles larger than about 250 μm can also be delivered from the devices, with the upper limitation being the point at which the size of the particles would cause untoward damage to the skin cells. The actual distance which the delivered particles will penetrate a target surface depends upon particle size (e.g., the nominal particle diameter assuming a roughly spherical particle geometry), particle density, the initial velocity at which the particle impacts the surface, and the density and kinematic viscosity of the targeted skin tissue. In this regard, optimal particle densities for use in needleless injection generally range between about 0.1 and 25 g/cm³, preferably between about 0.9 and 1.5 g/cm³, and injection velocities generally range between about 100 and 3,000 m/sec, or greater. With appropriate gas pressure, particles having an average diameter of 10-70 μm can be accelerated through the nozzle at velocities approaching the supersonic speeds of a driving gas flow.

If desired, these needleless syringe systems can be provided in a preloaded condition containing a suitable dosage of the particles comprising the antigen of interest and/or the selected adjuvant. The loaded syringe can be packaged in a hermetically sealed container, which may further be labelled as described above.

Compositions containing a therapeutically effective amount of the powdered molecules described herein can be delivered to any suitable target tissue via the above-described needleless syringes. For example, the compositions can be delivered to muscle, skin, brain, lung, liver, spleen, bone marrow, thymus, heart, lymph, blood, bone cartilage, pancreas, kidney, gall bladder, stomach, intestine, testis, ovary, uterus, rectum, nervous system, eye, gland and connective tissues. For nucleic acid molecules, delivery is preferably to, and the molecules expressed in, terminally differentiated cells; however, the molecules can also be delivered to non-differentiated, or partially differentiated cells such as stem cells of blood and skin fibroblasts.

The powdered compositions are administered to the subject to be treated in a manner compatible with the dosage formulation, and in an amount that will be prophylactically and/or therapeutically effective. The amount of the composition to be delivered, generally in the range of from 0.5 μg/kg to 100 μg/kg of nucleic acid molecule per dose, depends on the subject to be treated. Doses for other pharmaceuticals, such as physiological active peptides and proteins, generally range from about 0.1 μg to about 20 mg, preferably 10 μg to about 3 mg. The exact amount necessary will vary depending on the age and general condition of the individual to be treated, the severity of the condition being treated, the particular preparation delivered, the site of administration, as well as other factors. An appropriate effective amount can be readily determined by one of skill in the art.

Thus, a “therapeutically effective amount” of the present particulate compositions will be sufficient to bring about treatment or prevention of disease or condition symptoms, and will fall in a relatively broad range that can be determined through routine trials.

Eliciting Immune Responses

In another embodiment of the invention, a method for eliciting an immune response against B. anthracis in a subject is provided. In essence, the method entails providing a polynucleotide vaccine composition, where the compositions contains a nucleic acid molecule encoding a B. anthracis antigen, preferably the PA antigen. The nucleic acid sequence encoding the B. anthracis antigen is linked to regulatory sequences to provide an expression cassette. This expression cassette is then provided in a suitable vector, for example a plasmid vector construct. In particular embodiments, the B. anthracis antigen is substantially the full-length B. anthracis PA polypeptide, or a functional homologue thereof.

In one aspect, the method entails administering the vaccine composition to the subject using standard gene delivery techniques that are known in the art. See, e.g., U.S. Pat. Nos. 5,399,346, 5,580,859, 5,589,466. Typically, the polynucleotide vaccine composition is combined with a pharmaceutically acceptable excipient or vehicle to provide a liquid preparation (as described herein above) and then used as an injectable solution, suspension or emulsion for administration via parenteral, subcutaneous, intradermal, intramuscular, intravenous injection using a conventional needle and syringe, or using a liquid jet injection system. It is preferred that the composition be administered to skin or mucosal tissue of the subject. Liquid preparations can also be administered topically to skin or mucosal tissue, or provided as a finely divided spray suitable for respiratory or pulmonary administration. Other modes of administration include oral administration, suppositories, and active or passive transdermal delivery techniques. The polynucleotide vaccine compositions can alternatively be delivered ex vivo to cells derived from the subject, whereafter the cells are reimplanted in the subject. Upon introduction into the subject, the nucleic acid sequence is expressed to provide B. anthracis antigen in situ in an amount sufficient to elicit an anti-B. anthracis immune response in the vaccinated subject. This immune response can be a humoral (antibody) response, a cellular (CTL) response, or be characterized as raising both a humoral and a cellular immune response against the B. anthracis antigen.

It is preferred, however, that the polynucleotide vaccine composition be delivered in particulate form. For example, the vaccine composition can be administered using a particle acceleration device which fires nucleic acid-coated microparticles into target tissue, or transdermally delivers particulate nucleic acid compositions. In this regard, particle-mediated nucleic acid immunization has been shown to elicit both humoral and cytotoxic T lymphocyte immune responses following epidermal delivery of nanogram quantities of DNA. Pertmer et al. (1995) Vaccine 13:1427-1430. Particle-mediated delivery techniques have been compared to other types of nucleic acid inoculation, and found markedly superior. Fynan et al. (1995) Int. J. Immunopharmacology 17:79-83, Fynan et al. (1993) Proc. Natl. Acad. Sci. USA 90:11478-11482, and Raz et al. (1994) Proc. Natl. Acad. Sci. USA 91:9519-9523. Such studies have investigated particle-mediated delivery of nucleic acid-based vaccines to both superficial skin and muscle tissue.

As described in detail herein above, particle-mediated methods for delivering nucleic acid preparations are known in the art. Thus, the polynucleotide vaccine composition can be coated onto core carrier particles using a variety of techniques known in the art. Carrier particles are selected from materials which have a suitable density in the range of particle sizes typically used for intracellular delivery from a particle acceleration device. The optimum carrier particle size will, of course, depend on the diameter of the target cells.

These methods can alternatively be modified by coadministration of additional or ancillary components to the subject. For example, a suitable adjuvant component can be added to the polynucleotide vaccine composition or administered along with the vaccine composition. In addition, a secondary vaccine composition can be administered, wherein the secondary composition can comprise a further nucleic acid vaccine, e.g., a polynucleotide encoding an additional B. anthracis antigen derived or obtained from an B. anthracis LF or EF gene product, or the secondary vaccine composition can comprise a conventional B. anthracis vaccine such as the AVA commercial (recombinant subunit) anthrax vaccine. The secondary vaccine composition can be combined with the polynucleotide vaccine composition to form a single composition, or the secondary vaccine composition can be administered separately to the same or to a different site, either concurrently, sequentially, or separated by a significant passage of time such as in a boosting step some days after the initial vaccine composition has been administered.

As above, the secondary vaccine composition and/or the adjuvant component can be administered by injection using either a conventional syringe, or using a particle-mediated delivery system as also described above. Injection will typically be either subcutaneously, epidermally, intradermally, intramucosally (e.g., nasally, rectally and/or vaginally), intraperitoneally, intravenously, orally or intramuscularly. Other modes of administration include topical, oral and pulmonary administration, suppositories, and transdermal applications. Dosage treatment may be a single dose schedule or a multiple dose schedule.

In another aspect, the method entails transfecting cells of the subject with a polynucleotide vaccine composition that includes one or more recombinant nucleic acid molecules having a sequence or sequences encoding one or more B. anthracis antigens, preferably the PA antigen (as described herein above). The transfection is carried out under conditions that permit expression of the B. anthracis antigen in the subject. Expression of the B. anthracis antigen in situ is sufficient to elicit a protective immune response against B. anthracis. Transfection is effected using any of the above-described gene delivery techniques, with particle-mediated delivery being preferred. In addition, any of the secondary compositions, vaccine, adjuvant, or combinations thereof, can be used as described above.

### EXPERIMENTAL

Below are examples of specific embodiments for carrying out the present invention. The examples are offered for illustrative purposes only, and are not intended to limit the scope of the present invention in any way.

Efforts have been made to ensure accuracy with respect to numbers used (e.g., amounts, temperatures, etc.), but some experimental error and deviation should, of course, be allowed for.

### Example 1

Plasmid Construction

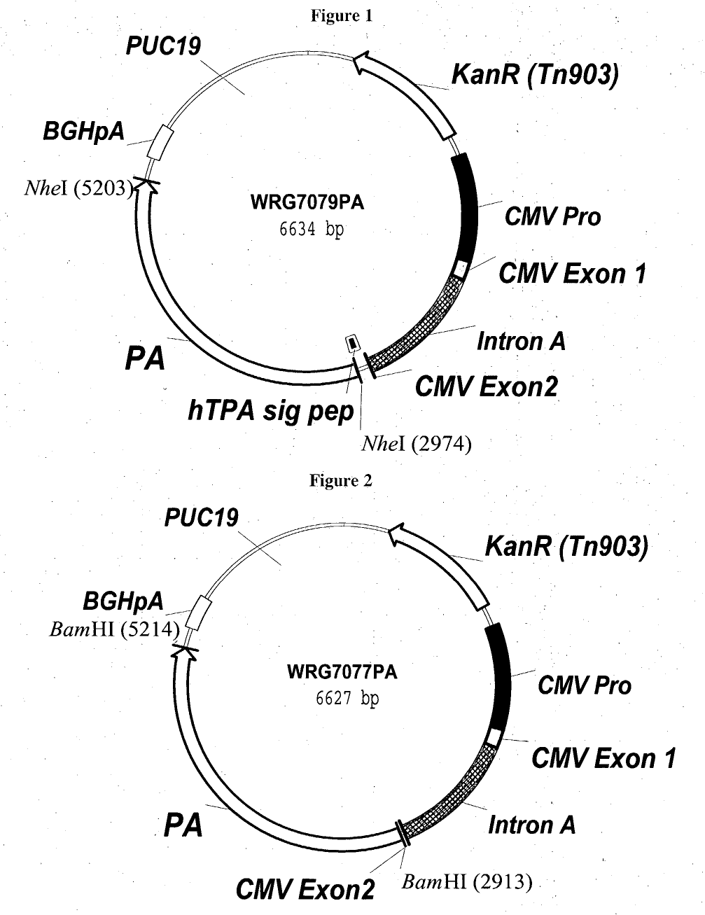

FIGS. 1A-1F depict the complete nucleotide sequence (SEQ ID NO:3) and the predicted amino acid sequence (SEQ ID NO:4) for the Bacillus anthracis PA antigen. The PA antigen sequence is also present in the Bluescript plasmid construct (Iacono-Connors, et al. (1990) Infection and Immunity 5:366-372). Accordingly, for the purposes of the following studies, the PA antigen encoding sequence was either cut from the Bluescript plasmid vector for subsequent insertion into the pWRG7077 plasmid vector, or the nucleotide sequence for the PA antigen was used as a model to design polymerase chain reaction (PCR) primers to facilitate cloning of the PA coding sequence into the pWRG7079 plasmid vector as follows.

1. The pWRG7077PA Plasmid Construct.

The pWRG7077 plasmid construct has been previously described (Schmaljohn et al. (1997) J. Virol. 71:9563-9569) and contains the immediate early promoter from human cytomegalovirus (hCMV) and its associated intron A sequence. This vector also includes a polyadenylation sequence from the bovine growth hormone gene. The pWRG7077 construct was obtained from PowderJect Vaccines, Inc., of Madison Wis. (formerly doing business as Auragen, Inc.).

The Bluescript plasmid construct (Iacono-Connors, et al. (1990) Infection and Immunity 5:366-372) was cleaved in order to generate a restriction fragment containing the full-length PA coding sequence clone suitable for insertion into the pWRG7077 plasmid. The approximately 1.2 kB fragment was isolated by gel electrophoresis and then digested with BamHI in order to generate a suitable insertion fragment, which was in turn inserted into the BamHI-cleaved pWRG7077 vector (at the BamHI site appearing at position 2919 in the plasmid), resulting in the pWRG7077PA expression vector. The pWRG7077PA expression construct contains the PA antigen sequence operatively linked to the CMV promoter and bovine growth hormone polyadenylation control sequences. A functional map of this vector is shown in FIG. 2.

1. The pWRG7079PA Plasmid Construct.

The pWRG7077 plasmid construct (Schmaljohn et al. (1997) J. Virol. 71:9563-9569) was cut with SalI and BamHI in order to remove a fragment containing the CMV promoter sequence and polylinker region. The resulting vector construct was termed pAB. Next, the pWRG7054 plasmid construct (PowderJect Vaccines, Inc., Madison, Wis., formerly doing business as Auragen, Inc.) was obtained. The pWRG7054 cloning vector contains the human cytomegalovirus immediate early promoter with the associated intron A sequence. In addition, the coding sequence for the signal peptide of human tissue plasminogen activator is included in pWRG7054 in order to allow for the secretion from mammalian cells of any protein whose coding sequence is inserted at the NheI site in the appropriate reading frame. (See, e.g., Chapman et al. (1991) Nuc. Acids Res. 19:3979-3986, and Burke et al. (1986) J. Biol. Chem. 261:12574-12578). The pWRG7054 construct was cut with SalI and BamHI to create an insertion fragment containing the CMV promoter and the TPA leader sequence, which was then inserted into pAB, thereby restoring the promoter and adding the TPA signal peptide sequence, resulting in the pWRG7079 cloning vector.

Next, a pair (5′ and 3′) PCR primers were designed and used to generate a PA coding sequence clone without the PA signal peptide sequence so that it was suitable for insertion into the pWRG7079 cloning vector. The primers were:

Forward PCR Primer:

5′-GTC AGC TAG CGA GGT GAT TCA GGC AGA AGT T-3′ (SEQ ID NO:5)

Reverse PCR Primer:

5′-CAG TGC TAG CTC CTA TCT CAT AGC C-3′ (SEQ ID NO:6).

PCR products were electrophoresed on a 2% agarose gel revealing a single DNA band of the expected size of approximately 1.2 kB. This band was isolated from the gel and digested with NheI in order to generate the necessary sticky ends for insertion into the pWRG7079 cloning vector. The pWkG7079 DNA was digested with NheI to facilitate insertion of the PA coding insert into the NheI site appearing at position 2974 in the plasmid. The resulting PA expression vector was termed pWRG7079PA. A functional map of this vector is shown in FIG. 2. The pWRG7079PA vector contains the immediate early promoter from human cytomegalovirus (hCMV) and its associated intron A sequence to drive transcription from the PA coding sequence, as well as a polyadenylation sequence from the bovine growth hormone gene. The construct further contains the human tissue plasminogen activator (hTPA) signal peptide. The PA antigen sequence was inserted in-frame with the TPA signal sequence so as to provide for efficient secretion of the PA antigen from mammalian cells.

The sequence for the TPA secretion signal peptide is depicted below as SEQ ID NO:7 .

ATG GAT GCA ATG AAG AGA GGG CTC TGC TGT GTG CTG CTG CTG TGT GGA GCA GTC TTC GTT TCG GCT (SEQ ID NO:7).

The protein sequence for this TPA signal peptide is depicted below as SEQ ID NO:8.

MDAMKRGLCC VLLLCGAVFV SA (SEQ ID NO:8).

Once produced, the pWRG7077PA and pWRG7079PA plasmid constructs were used to immunize animals in the following experiments.

### Example 2

Induction of PA-Specific Antibody Responses Guinea Pigs

The following study was carried out in order to assess the ability to generate anti-PA antibody responses using the nucleic acid immunization techniques of the present invention. In addition, the ability to protect against a lethal Bacillus anthracis challenge was also assessed.

Coating the Core Carrier Particles: Appropriate weights of gold particles were weighted directly into 1.5 mL Eppendorf tubes. 400-500 μL of a 0.05M spermidine was then added, and clumps of gold in the gold/spermidine solution were broken-up using a water bath sonicator for 3-5 seconds. DNA stock solution, containing the pWRG7079PA plasmid construct, was added to the gold/spermidine solution to result in a bead loading rate of 2.0 μg DNA/mg Au, and the tubes were capped and inverted to mix, then vortexed briefly. After adjusting the vortex speed down, and while vortexing gently, a volume of 10% CaCl₂ was added dropwise to an amount equal to the volume of spermidine added to the dry gold. Once the entire volume of CaCl₂ was added, the resultant solution was vortexed at high speed for about 5 seconds. The solution was then allowed to precipitate at room temperature for at least 10 minutes. After the ten minute precipitation, the tubes were centrifuged briefly (10-15 seconds) to pellet all of the gold. The supernatant was aspirated, and the tubes were “raked” across an Eppendorf rack to loosen the gold pellet. 800 μL of EtOH was added, and the tubes were inverted several times to wash the DNA-coated gold. This step was repeated twice, after which the tubes were again centrifuged and the supernatant aspirated. The washed DNA-coated gold particles were then loaded into lengths of Tefzel™ tubing as previously described. See e.g., PCT patent application PCT/US95/00780 and U.S. Pat. Nos. 5,733,600; 5,780,100; 5,865,796 and 5,584,807, the disclosures of which are hereby incorporated by reference.

Experimental Groups: The following experimental groups of guinea pigs were assembled: Group 1=16 animals inoculated 4 times (at weeks 0, 4, 8 and 12) with the AVA vaccine product administered via intramuscular injection; Group 2=16 animals inoculated 4 times (at weeks 0, 4, 8 and 12) with the pWRG7079PA DNA vaccine; Group 3=8 animals inoculated with a saline+alhydrogel control 4 times (at weeks 0, 4, 8 and 12); and Group 4=8 animals inoculated 4 times (at weeks 0, 4, 8 and 12) with an empty plasmid vector (pWRG7079) control.

The Group 2 and Group 4 animals received particle-mediated DNA immunizations at four week intervals in which each immunization consisted of particle-mediated deliveries of DNA coated gold particles using a PowderJect XR-1 particle acceleration device (PowderJect Vaccines, Inc., Madison, Wis.) at a helium pressure of 400 p.s.i.

Blood samples were collected and the sera was analyzed for PA-specific antibody responses using a standard ELISA assay in which ELISA plates were pre-coated with a purified PA peptide.

At the end of the vaccination scheme, all animals were challenged (at 16 weeks) by intramuscular injection of 1×10⁴ Ames spores. The anti-PA sera titers and % survival in the vaccinated animals are reported below in Table 1.

TABLE 1

| | Survival/ | | | |
|---|---|---|---|---|
| Vaccine Group | Total | % Survival | GM Titer* | Titer Range |
| AVA (Group 1) | 6/16 | 38% | 24514 | 12800-25600 |
| pWRG7079PA | 0/16 | 0% | 1745 | 400-12800 |
| (Group 2) | | | | |
| Alhydrogel + | 0/16 | 0% | 10 | 10 |
| Saline (Group 3) | | | | |
| pWRG7079 | 0/16 | 0% | 10 | 10 |
| (Group 4) | | | | |

As can be seen, although the PA DNA vaccine composition was able to generate an antibody response in the vaccinated animals, it was not found to be protective against the Bacillus anthracis challenge. In similar manner, the AVA vaccine was only able to provide partial protective immunity, wherein 38% of the vaccinated animals were protected against the lethal challenge. It is now generally regarded that the guinea pig animal model is a poor predictor for human disease.

### Example 3

Induction of PA-Specific Antibody Responses Rabbits

The following study was carried out in order to assess the ability to generate anti-PA antibody responses using the nucleic acid immunization techniques of the present invention. In addition, the ability to protect against a lethal Bacillus anthracis challenge was also assessed. An in vitro correlate of immunity in a rabbit model of inhalational anthrax has recently been reported, where a strong serological correlate of vaccine-induced immunity has been established. Pitt et al. (2001) Vaccine 19:4768-4773. In contrast to the guinea pig animal model system, the rabbit model proposed by Pitt et al. is a very good predictor of human disease. Accordingly, the experiment of Example 2 was repeated using a new rabbit model as follows.

Coating the Core Carrier Particles: Appropriate weights of gold particles were weighted directly into 1.5 mL Eppendorf tubes. 400-500 μL of a 0.05M spermidine was then added, and clumps of gold in the gold/spermidine solution were broken-up using a water bath sonicator for 3-5 seconds. DNA stock solution, containing the pWRG7079PA plasmid construct, was added to the gold/spermidine solution to result in a bead loading rate of 2.0 μg DNA/mg Au, and the tubes were capped and inverted to mix, then vortexed briefly. After adjusting the vortexer speed down, and while vortexing gently, a volume of 10% CaCl₂ was added dropwise to an amount equal to the volume of spermidine added to the dry gold. Once the entire volume of CaCl₂ was added, the resultant solution was vortexed at high speed for about 5 seconds. The solution was then allowed to precipitate at room temperature for at least 10 minutes. After the ten minute precipitation, the tubes were centrifuged briefly (10-15 seconds) to pellet all of the gold. The supernatant was aspirated, and the tubes were “raked”across an Eppendorf rack to loosen the gold pellet. 800 μL of EtOH was added, and the tubes were inverted several times to wash the DNA-coated gold. This step was repeated twice, after which the tubes were again centrifuged and the supernatant aspirated. The washed DNA-coated gold particles were then loaded into lengths of Tefzel# tubing as previously described. See e.g., PCT patent application PCT/US95/00780 and U.S. Pat. Nos. 5,733,600; 5,780,100; 5,865,796 and 5,584,807, the disclosures of which are hereby incorporated by reference.

Experimental Groups: The following experimental groups (New Zealand white rabbits) were assembled: Group 1=10 animals inoculated 3 times (at weeks 0, 4 and 8) with the AVA vaccine product, given at 0.5 mL by intramuscular injection into the caudal thigh muscle; Group 2=10 animals inoculated 3 times (at weeks 0, 4 and 8) with the pWRG7079PA DNA vaccine, where each inoculation consisted of 8 shots to administer a total of 20 μg DNA/animal; and Group 3=10 animals inoculated 3 times (at weeks 0, 4 and 8) with an empty plasmid vector control (the pWRG7079 plasmid), where each inoculation consisted of 8 shots to administer a total of 20 μg DNA/animal. For the Group 2 and Group 3 animals gold/DNA deliveries were accomplished using a PowderJect XR-1 particle acceleration device (PowderJect Vaccines, Inc., Madison, Wis.) at a helium pressure of 400 p.s.i.

Blood samples were collected at the following time points: week -1 (prebleed); week 4; week 8, week 12, week 17, week 21 and week 25. Sera were analyzed for PA-specific antibody responses using a standard ELISA assay. More particularly, 96-well ELISA plates were pre-coated with a purified PA peptide obtained from SAIC, Inc. (Fort Detrick, Md.) and incubated overnight at 4° C. The plates were then washed five times with PBST. Pooled sera were started at a 1:25 dilution antibody in block buffer (5% milk in PBST), and serially diluted at 1:4 across the plates. The plates were then incubated at 37° C. for one hour, after which the plates were washed five times with PBST. Goat anti-rabbit-HRP secondary antibody was diluted 1:1000, and 100 μL added to each well, after which the pates were incubated at 37° C. for one hour. The plates were then washed five times using PBST, and stained using 100 μL of ABTS reagent (warmed to room temperature). The plates were incubated at room temperature for 30 minutes, after which time the color development reaction was stopped using 100 μL of the ABTS stop solution and the plates were read at 450 nm.

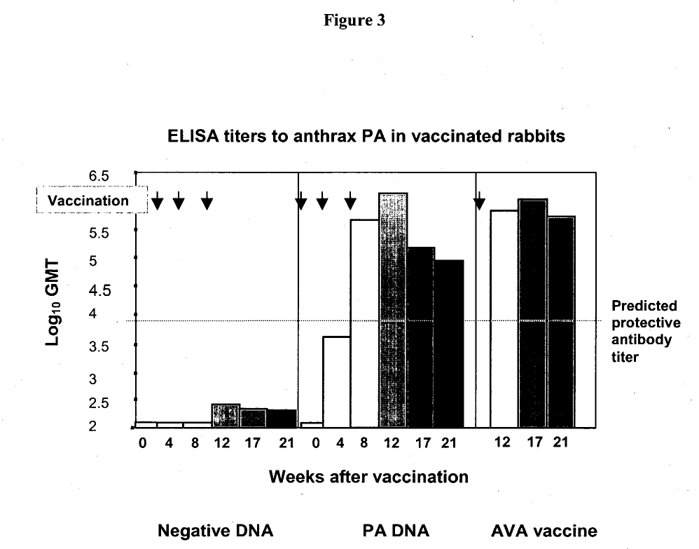

At 12 weeks after the third inoculation, all animals received a single booster just prior to a subcutaneous challenge with 1×10⁴ Ames spores. The anti-PA sera titers are depicted in FIG. 3, and the % survival data in the vaccinated animals are reported below in Table 2.

TABLE 2

| Vaccine Group | Survival/Total | % Survival |
|---|---|---|
| AVA (Group 1) | 7/10 | 70% |
| pWRG7079PA (Group 2) | 9/10 | 90% |
| pWRG7079 (Group 3) | 0/10 | 0% |

As can be seen in Table 2, while all control animals died, 90% survival was seen in the PA DNA vaccine test group as compared with 70% survival in the AVA-vaccinated group.

Accordingly, novel recombinant nucleic acid molecules, compositions comprising those molecules, and nucleic acid immunization techniques have been described. Although preferred embodiments of the subject invention have been described in some detail, it is understood that obvious variations can be made without departing from the spirit and the scope of the invention as defined by the appended claims.

What is claimed is:

1. A polynucleotide vaccine composition comprising a nucleic acid sequence that encodes a Bacillus anthracis antigen, wherein said nucleic acid sequence is operatively linked to a promoter suitable for expression of the antigen in a mammalian cell.

2. The composition of claim 1 wherein the nucleic acid sequence is present in a plasmid vector.

3. The composition of claim 1 wherein the nucleic acid sequence encodes an antigen obtained or derived from the Protective Antigen of Bacillus anthracis.

4. The composition of claim 3 wherein the antigen encoded by the nucleic acid sequence is substantially homologous to the full-length Protective Antigen protein.

5. A polynucleotide vaccine composition, said composition comprising: a first nucleic acid sequence that encodes a Bacillus anthracis antigen; and a second nucleic acid sequence that encodes a leader signal peptide operatively linked to the first nucleic acid sequence, wherein said first and said second nucleic acid sequences are operatively linked to a promoter suitable for expression thereof in a mammalian cell and said leader signal peptide provides for the secretion of the encoded antigen.

6. The composition of claim 1 further comprising an adjuvant component.

7. The composition of claim 6 wherein said adjuvant component is present in the composition in the form of a nucleic acid sequence.

8. The composition of claim 7 wherein said adjuvant component is a CpG sequence.

9. The composition of claim 7 wherein said adjuvant component is a further nucleic acid sequence that encodes a polypeptide adjuvant.

10. The composition of claim 6 wherein said adjuvant component is present in the composition in a form other than a nucleic acid sequence.

11. The composition of claim 10 wherein said adjuvant component is selected from the group consisting of a polypeptide, a lipid, a non-protein hormone, and a vitamin.

12. The composition of claim 11 wherein the adjuvant component comprises monophosphoryl lipid A.

13. The composition of claim 11 wherein the adjuvant component comprises a saponin or a derivative thereof.

14. The composition of claim 13 wherein the adjuvant component comprises Quil-A.

15. The composition of claim 1 further comprising a pharmaceutically acceptable excipient or vehicle.

16. The composition of claim 1 wherein said composition is in particulate form.

17. The composition of claim 16 wherein the nucleic acid sequence is coated onto a core carrier particle.

18. The composition of claim 17 wherein the core carrier particle has an average diameter of about 0. to about 10 μm.

19. The composition of claim 17 wherein the core carrier particle comprises a metal.

20. The composition of claim 19 wherein the metal is gold.

21. The composition of claim 1 further comprising a transfection facilitating agent.

22. The composition of claim 5 further comprising an adjuvant component.

23. The composition of claim 22 wherein said adjuvant component is present in the composition in the form of a nucleic acid sequence.

24. The composition of claim 23 wherein said adjuvant component is a CpG sequence.

25. The composition of claim 23 wherein said adjuvant component is a further nucleic acid sequence that encodes a polypeptide adjuvant.

26. The composition of claim 5 wherein said adjuvant component is present in the composition in a form other than a nucleic acid sequence.

27. The composition of claim 26 wherein said adjuvant component is selected from the group consisting of a polypeptide, a lipid, a non-protein hormone, and a vitamin.

28. The composition of claim 27 wherein the adjuvant component comprises monophosphoryl lipid A.

29. The composition of claim 27 wherein the adjuvant component comprises a saponin or a derivative thereof.

30. The composition of claim 29 wherein the adjuvant component comprises Quil-A.

31. The composition of claim 5 further comprising a pharmaceutically acceptable excipient or vehicle.

32. The composition of claim 5 wherein said composition is in particulate form.

33. The composition of claim 32 wherein the nucleic acid sequence is coated onto a core carrier particle.

34. The composition of claim 33 wherein the core carrier particle has an average diameter of about 0.1 to about 10 μm.

35. The composition of claim 34 wherein the core carrier particle comprises a metal.

36. The composition of claim 35 wherein the metal is gold.

37. The composition of claim 5 further comprising a transfection facilitating agent.

38. A method for eliciting an immune response against Bacillus anthracis in a subject, the method comprising administering the vaccine composition of claim 5 to the subject, whereby upon introduction to the subject, the nucleic acid sequence is expressed to provide the Bacillus anthracis antigen in an amount sufficient to elicit said immune response.

39. The method of claim 38 wherein the vaccine composition is administered directly into skin or muscle tissue.

40. The method of claim 38 wherein the vaccine composition is administered to the subject in particulate form.

41. The method of claim 38 wherein the nucleic acid sequence is coated onto a core carrier particle and administered to the subject using a particle-mediated delivery technique.

42. The method of claim 38 wherein the vaccine composition further comprises an adjuvant component.

43. The method of claim 38 further comprising the step of administering a second vaccine composition to the subject.

44. The method of claim 43 wherein the second vaccine composition is an anti-Bacillus anthracis vaccine containing the peptide form of the Protective Antigen from Bacillus anthracis.

45. The method of claim 43 wherein the second vaccine composition is administered to the subject in a boosting step.

46. The method of claim 43 wherein both vaccine compositions are administered to the same site in the subject.

47. The method of claim 43 wherein the vaccine compositions are administered concurrently.

48. The method of claim 43 wherein the vaccine compositions are combined to provide a single composition.

49. A method for using a Bacillus anthracis antigen to induce a protective immune response in a subject, said method comprising:
(a) providing an expression cassette containing a nucleic acid sequence encoding the Protective Antigen from Bacillus anthracis operatively linked to control sequences that direct expression of the Protective Antigen when introduced into tissue of the subject; and
(b) administering the expression cassette to tissue of the subject such that the Protective Antigen is expressed in an amount sufficient to induce said protective immune response in the subject.

50. The method of claim 49 wherein the expression cassette is present in a plasmid vector.

51. A method for using a Bacillus anthracis antigen to induce an immune response in a subject, said method comprising:
(a) providing an expression cassette containing a first nucleic acid sequence encoding the Protective Antigen from Bacillus anthracis and a second nucleic acid sequence that encodes a leader signal peptide, wherein said first and second nucleic acid sequences are operatively linked to each other and to control sequences that direct expression of said sequences when introduced into tissue of the subject and said leader signal peptide provides for the secretion of the encoded Protective Antigen; and
(b) administering the expression cassette to tissue of the subject such that the Protective Antigen is expressed in an amount sufficient to induce said immune response in the subject.

52. The method of claim 51 wherein the leader signal peptide is the tissue plasminogen activator (TPA) leader signal peptide.

53. The method of claim 51 wherein the expression cassette is present in a plasmid vector.

54. The method of claim 53 wherein the plasmid vector is administered directly into skin or muscle tissue of the subject.

55. The method of claim 53 wherein the plasmid vector is administered to the subject in particulate form.

56. The method of claim 55 wherein the plasmid vector is coated onto a core carrier particle and administered to the subject using a particle-mediated delivery technique.
